(19) United States
(12) Patent Application Publication
(10) Pub. No.: US 2005/0003378 A1
(43) Pub. Date: Jan. 6, 2005

(54) Inhibitor of demethylase, antitumorigenic agent, and an in vitro assay for demethylase inhibitors

(76) Inventor: Moshe Szyf, Cote St. Luc. (CA)

Correspondence Address: KLAUBER & JACKSON, 411 HACKENSACK AVENUE, HACKENSACK, NJ, 07601, US

(21) Appl. No.: 10/738,634

(22) Filed: Dec. 17, 2003

Related U.S. Application Data
(60) Provisional application No. 60/434,397, filed on Dec. 19, 2002.

(51) Int. Cl. C12Q 1/68; A61K 48/00

(52) U.S. Cl. 435/6; 514/46

(57) ABSTRACT

The present invention relates to a DNA demethylase (dMTase) inhibitor which comprises s-adenosylmethionine, a metabolite of s-adenosylmethionine or a pharmaceutically acceptable salt thereof.

### CROSS REFERENCE TO RELATED APPLICATIONS

The present application claims priority from U.S. Provisional Application Ser. No. 60/434, 397 filed Dec. 15, 2002. Applicants claim priority under 35 U.S.C. § 119 as to the said U.S. application, and the entire disclosure of this application is incorporated herein in its entirety.

### BACKGROUND OF THE INVENTION

(a) Field of the Invention

The invention relates to an in vitro assay for demethylase inhibitors, demethylase inhibitor and an antitumorigenic agent.

(b) Description of Prior Art

S-Adenosylmethionine (AdoMet) is the main methyl donor in numerous methyltransferase reactions in all organisms. The reduced derivative of 5,10-methylenetetrahydrofolate, 5-methyltetrahydrofolate, provides the methyl group for methionine and AdoMet synthesis. A series of rodent experiments as well as epidemiological data have suggested a correlation between low methyl-group and folate dietary intake and the risk for colorectal adenomas and cancer. Methyl deficient diets have been shown to promote liver cancer in rodents (1) and, AdoMet treatment was shown to prevent the development of liver cancer in rat (2).

In light of the clinical and epidemiological data suggesting a link between AdoMet levels and cancer, it is important to understand the tumor protective mechanism of action of AdoMet, as well as the tumor promoting action of methyl deficient diets. This is of importance not only for realizing the therapeutic potential of this natural vitamin, but also for unraveling basic mechanisms of tumorigenesis, especially the role of methyl group metabolism. AdoMet is the cofactor for transmethylation reactions including DNA methylation (3, 4), while S-adenosylhomocysteine (AdoHcy) is the product of transmethylation reactions and an inhibitor of DNMTs. A current model is that exogenous administration of AdoMet increases the intracellular ratio of AdoMet to AdoHcy, thus stimulating DNMT activity resulting in increased DNA methylation (2). An increase in AdoHcy concentrations, even without a concomitant reduction in AdoMet results in inhibition of DNMT and DNA hypomethylation. Methyl deficient diets decrease intracellular AdoMet concentration; increase AdoHcy concentrations and trigger DNA hypomethylation (1). A genetic link was established between polymorphisms in the Methylenetetrahydrofolate Reductase gene encoding the enzyme catalyzing the synthesis of 5-methyltetrahydrofolate, and DNA hypomethylation. Global hypomethylation of DNA is a hallmark of cancer (5). If the mechanism of action of methyl-rich diets in cancer chemoprevention and methyl-deficient diet in cancer promotion is through changing genomic methylation status, then it implies that global hypomethylation plays a causal role in cancer. This hypothesis is supported by the observation that 5-azaC, a DNMT inhibitor can reverse AdoMet mediated chemoprevention of liver carcinogenesis (6).

Although it has been controversial, there is now little doubt that exogenous AdoMet increases the intracellular AdoMet levels. AdoMet uptake into cells has also been verified through a high performance liquid chromatography analysis (7). A number of data support the notion that exogenous AdoMet causes hypermethylation of DNA (8).

Whereas this model provides an attractively simple explanation as to the possible relationship between exogenous AdoMet administration and DNA methylation, there are a number of unresolved issues. First, increased AdoMet should increase DNMT activity only if the normal intracellular concentration of AdoMet is below the Km for the enzyme, this has not been demonstrated as of yet. It is however possible that the main mechanism by which elevating AdoMet levels increases DNMT activity is by competing with AdoHcy, an inhibitor of DNMT. Such an indirect mechanism of activation might be relevant even if the basal level of AdoMet is above the Km. Second, even if exogenous administration of AdoMet increases the activity of DNMT, it is not clear whether the normal level of DNMT activity is the limiting factor, or whether the specificity of DNA methylation patterns is determined by the rate of DNMT activity. There is no evidence to suggest that specific sites remain unmethylated in vertebrate genomes simply for the reason that the rate of DNMT activity is limiting. Third, methyl deficient diets cause hypomethylation in the liver whose cells are mostly postmitotic and do not replicate (1). If the mechanism of this hypomethylation involves only inhibition of DNMT, it could take effect only in cells that actively synthesize DNA. Since measurable demethylation is seen with these diets, this could only occur if a significant fraction of liver cells proliferate during the treatment. Whereas an increase in proliferation in the liver is seen as a consequence of methyl deficient diets, it is not clear whether proliferation precedes or follows global hypomethylation.

The current hypothesis on the mechanism of action of AdoMet is based on the assumption that DNA methylation is a unidirectional and irreversible reaction, which is catalyzed by DNMT exclusively. However, an increasing list of data supports the hypothesis that DNA methylation in vivo is also fashioned by demethylase activity. A DNA demethylase activity was partially purified from human lung carcinoma A549 cells (9) and the protein MBD2b was shown to bear demethylase activity (10). The demethylase activity of MBD2 was disputed by several groups (11), but our recent data demonstrated that ectopic MBD2/dMTase causes DNA demethylation in a promoter specific manner (12). We therefore proposed that DNA methylation is a reversible reaction, and that the steady state DNA methylation status of a gene reflects a balance of methylation and demethylation (13, 14). Thus, it is possible that AdoMet increases methylation by inhibiting demethylation.

It would be highly desirable to be provided with an in vitro assay for demethylase inhibitors.

It would be highly desirable to be provided with a demethylase inhibitor and an antitumorigenic agent.

### SUMMARY OF THE INVENTION

One aim of the present invention is to provide an in vitro assay for demethylase inhibitors.

Another aim of the present invention is to provide AdoMet as a DNA hypermethylating or demethylase inhibitor, and an antitumorigenic agent.

It is impossible to determine whether hypermethylation of a gene in vivo following chronic drug treatment is caused by either an increase in DNMT activity, as proposed by the current model, or inhibition of demethylase. A model system is required to study either methylation or demethylation in isolation from the reverse activity. We have recently described such a model (FIG. 1). When an unmethylated CMV-GFP plasmid is transiently transfected into the human embryonal kidney cell line HEK 293, it remains unmethylated throughout the transient transfection period up to 96 hours, demonstrating that DNMTs do not target extrachromosomal DNA under the conditions of our experiment (15). When an in vitro methylated CMV-GFP plasmid is transiently transfected into these cells, it generally remains methylated. However, when histone acetylation is induced by Trichostatin A (TSA), the plasmid is fully and actively demethylated by an endogenous demethylase activity. Since the plasmid does not replicate during the time frame of the experiment, this assay measures only active demethylation (15). This system thus allows us to determine the impact which different factors might have on demethylase activity. We have recently used this assay to illustrate that a protein that inhibits histone acetylation inhibits active demethylation in living cells (16).

In this paper we took advantage of this assay to test the hypothesis that exogenous administration of AdoMet inhibits demethylase activity in living cells. Using an in vitro demethylase assay, we then tested whether AdoMet inhibits recombinant MBD2/dMTase activity extracted from infected SF9 insect cells. We also show that concentrations of exogenous AdoMet which inhibit demethylase activity also inhibit the transformation potential of HEK 293 and A549 cells in vitro as determined by a soft agar assay. Taken together, our results support a new alternative hypothesis for the mechanism of action of AdoMet as a DNA hypermethylating and an antitumorigenic agent.

In accordance with the present invention there is provided a DNA demethylase (dMTase) inhibitor which comprises s-adenosylmethionine, a metabolite of s-adenosylmethionine or a pharmaceutically acceptable salt thereof.

The DNA demethylase is preferably methylated DNA binding protein 2/DNA demethylase (MBD2/dMTase), and more preferably comprises an amino acid sequence set forth in SEQ ID NOS: 1-4.

In accordance with the present invention there is provided an antitumorigenic agent which comprises s-adenosylmethionine, a metabolite of s-adenosylmethionine or a pharmaceutically acceptable salt thereof.

In accordance with the present invention there is provided a DNA hypermethylating agent which comprises s-adenosylmethionine, a metabolite of s-adenosylmethionine or a pharmaceutically acceptable salt thereof.

In accordance with the present invention there is provided a method for inhibiting a DNA demethylase (dMTase) comprising the steps of:
a) incubating a mixture comprising ³²P-prelabeled DNA, a demethylation buffer, purified MBD2/dMTase and s-adenosylmethionine and/or s-adenosylhomocysteine;
b) extracting the DNA from the MBD2/dMTase;
c) digesting the DNA with a microccocal nuclease, whereby obtaining ³²P-labeled 3′ mononucleotides;
d) purifying the labeled mononucleotides obtained in step (c); and
e) quantifying demethylation of the DNA..

In accordance with the present invention there is provided a method for inhibiting a tumorigenesis or a tumorigenic potential of cells of a patient which comprises the step of administering to the patient a medicament comprising s-adenosylmethionine, a metabolite of s-adenosylmethionine or a pharmaceutically acceptable salt thereof.

The medicament may be administered intravenously, intramuscularly, sub-cutaneously, transdermally, or per os.

The cells may be selected from the group consisting of A549 human lung carcinoma cells, and HEK 293 human embryonal kidney cells.

The patient is preferably a mammalian, more preferably a human.

In accordance with the present invention there is provided a method for inhibiting tumor cell growth of a patient which comprises the step of administering to the patient a medicament comprising s-adenosylmethionine, a metabolite of s-adenosylmethionine or a pharmaceutically acceptable salt thereof.

The tumor may be a liver tumor.

In accordance with the present invention there is provided an assay for identifying a DNA demethylase inhibitor (dMTase) which comprises the steps of:
a) incubating a mixture comprising ³²P-prelabeled DNA, a demethylation buffer, purified MBD2/dMTase and s-adenosylmethionine and/or s-adenosylhomocysteine;
b) extracting the DNA from the MBD2/dMTase;
c) digesting the DNA with a microccocal nuclease, whereby obtaining ³²P-labeled 3′ mononucleotides;
d) purifying the labeled mononucleotides obtained in step (c); and
e) quantifying demethylation of the DNA..

The assay buffer is preferably 10 mM Tris-HCl and 5 mM MgCl2, and more preferably has a pH of 7.0.

In step (b) of a preferred assay of the present invention, DNA is extracted from MBD2/dMTase by incubating a composition comprising the DNA, an extraction buffer and a proteinase K.

In step (b) of a preferred assay of the present invention, after the incubation, DNA is further extracted using a first organic solvent and tRNA, and the DNA is then precipitated using a salt and a second organic solvent.

The first organic solvent is preferably phenol or chloroform.

The second organic solvent is preferably ethanol.

In step (d) of a preferred assay of the present invention, the mononucleotides are purified by thin layer chromatography or column chromatography.

In step (e) of a preferred assay of the present invention, the labeled mononucleotides are visualized by autoradiography on a phosphorimager plate.

In accordance with the present invention there is provided an assay for identifying a method of high throughput screening of DNA demethylase (dMTase) inhibitors in living cells comprising the steps of:
a) transfecting HEK 293 cells in a 96 wells dish with methylated CMV-GFP;
b) treating wells with 300 μM TSA and subsequently adding inhibitors for screeing;
c) reading fluorescence to determine inhibition of. DNA demethylase, wherein a putative inhibitor shows dose dependent inhibition of fluorescence.

A preferred assay further comprises a step, conducted after step (c), extracting DNA from wells with low fluorescence to validate putative inhibitor activity.

### BRIEF DESCRIPTION OF THE DRAWINGS

FIG. 1 illustrates an outline of the model system used to assess the effects of AdoMet on active demethylation in living cells. CMV-GFP plasmid is methylated in vitro and transiently transfected into HEK 293 cells. Histone acetylation is induced with TSA, which results in DNA demethylation by endogenous demethylase activity (15). The degree of demethylation is then measured by methylation sensitive restriction digestion using HpaII enzyme which is followed by Southern Blot analysis using a GFP specific probe (AvaII-Cfr101 fragment). The possible mechanisms of action of AdoMet are indicated—either a direct inhibition of demethylase activitiy (1), or an indirect mechanism by first stimulating histone methylation and inhibiting histone acetylation, which would then inhibit active demethylation (2).

FIG. 2 illustrates that AdoMet inhibits active demethylation of CMV-GFP. A,B,C, Either in vitro methylated CMV-GFP plasmid (m-GFP) (A,B), or unmethylated CMV-GFP (GFP) (C) were transiently transfected into HEK 293 cells. Cells were treated with a final concentration of 0.3 μM TSA (+TSA), or left untreated (−TSA), and increasing concentrations of either AdoMet or AdoHcy (2, 4, 8 mM) were added. Cells were harvested 72 hours post transfection, and the methylation status of CMV-GFP was determined by MspI/HpaII restriction digestion and Southern Blot analysis as outlined in Materials and Methods and FIG. 1. M-methylated and HpaII undigested GFP; U-unmethylated and HpaII fully digested GFP (529 bp). D, The results of 3 independent experiments as shown in (A) were quantified by densitometry, and the average percent methylation remaining for each sample was calculated as outlined in Materials and Methods and charted ±S.D. E, The results of 3 independent experiments as shown in (B) were quantified as in (D) and the averages ±S.D. are presented.

FIG. 3 illustrates that AdoMet reduces TSA induced expression of methylated CMV-GFP. A,B, HEK 293 cells were transiently transfected with either methylated CMV-GFP plasmid (m-GFP) (A), or unmethylated CMV-GFP (GFP) (B), and treated with 0.3 μM TSA and with the indicated concentrations (mM) of AdoMet or AdoHcy. Total cell extracts were prepared using standard protocols and resolved on a 12.5% SDS-polyacrylamide gel. Proteins were transferred to PDVF membrane, and GFP protein was detected using rabbit polyclonal IgG (Santa Cruz, sc-8334). C, Experiments such as those shown in (A) were performed in triplicate and quantified by densitometry. Results are presented as arbitrary units relative to the level of GFP expression in the presence of TSA alone which was normalized to one. D, Quantification of triplicate experiments as shown in (B); the averages ±S.D. are presented.

FIG. 4 illustrates that AdoMet inhibits the growth of A549 and HEK 293 cells in soft agar. A549 cells (A) and HEK 293 cells (B) were treated with either 4 mM AdoMet or AdoHcy for 24 hours, or left untreated (control). 3×10³ live cells were then seeded in triplicate onto a six well dish in 4 ml of complete medium containing 0.33% agar solution. Colonies were counted 7 days after plating for HEK 293 cells, and 21 days after plating for A549 cells. Results shown are the average colony number per well from triplicate assays.

FIG. 5 illustrates Partial purification of His-MBD2/dMTase from SF 9 cells. A, Nuclear extracts of SF9 cells infected with MBD2/dMTase baculovirus were subjected to chromatography on Q-Sepharose and eluted with a step wise gradient of NaCl. Fractions were assayed for demethylase activity using a ³²P-prelabeled methylated DNA from micrococcus leisodeikticus. Demethylation activity elutes almost exclusively at 0.4 M NaCl with some activity present in the 0.2 M NaCl fraction. Following demethylation, the DNA was digested to 3′-mononucleotides and separated by thin layer chromatography. The DNA incubated with the whole cell extract (EX) and the flow through (FT) could not be recovered for activity analysis, most likely due to nuclease activities in the fractions. The MBD2/dMTase tightly binds to Q-sepharose as shown by the fact that the washes are free of demethylation activity. B, The fractions were analyzed by Western Blot using anti-Xpress antibody (Invitrogen) to demonstrate the presence of His-MBD2/dMTase (indicated by the arrow). The presence of the protein correlates with its activity in the elution profile, with almost all MBD2/dMTase detected at 0.4 M NaCl. Lower molecular weight bands in the extract and the bindings might be due to partial degradation of the protein during purification. NM, unmethylated control; M, methylated control; 5mC, 5-methyldeoxycytidine 3′-monophosphate; C, deoxycytidine 3′-monophosphate, EX, extract; FT, flow through.

FIG. 6 illustrates that AdoMet, but not AdoHcy, inhibits demethylation activity of MBD2/dMTase in vitro. A, ³²P-prelabeled methylated DNA from micrococcus leisodeikticus was incubated with the MBD2/dMTase and increasing concentrations of AdoMet. The autoradiography of one representative TLC plate is shown. B, Quantitative autoradiography of three independent experiments similar to the experiment shown in (A). Freeze dried AdoMet buffer was added in increasing volumes to a series of reactions as a control. C, Increasing concentrations of AdoHcy was added to the demethylase reaction; a representative experiment is shown. D, Quantification of the experiment in (C). E, Increasing concentrations of AdoHcy was added to reaction mixtures containing 10 mM AdoMet and demethylase activity was determined. NM, unmethylated control; M, methylated control; 5mC, 5-methyldeoxycytidine 3′-monophosphate; C, deoxycytidine 3′-monophosphate.

FIG. 7 illustrates possible model depicting how AdoMet may alter DNA methylation patterns and exert a chemoprotective effect. The steady state methylation pattern of a gene is determined by an equilibrium of DNMTs and DNA demethylases acting upon it. In cells where DNA demethylase is overexpressed, certain genes may have a tendency to become hypomethylated, and some of these genes may promote anchorage independent growth and tumorigenesis. In this case, the administration of AdoMet would have a tumor protective effect by inhibiting demethylation and shifting the equilibrium to the normally methylated state.

### DETAILED DESCRIPTION OF THE INVENTION

S-adenosylmethionine (AdoMet) is the methyl donor of numerous methylation reactions. Exogenous administration of AdoMet was shown to cause hypermethylation of DNA and to protect against liver disease and liver cancer in rodent models. In addition, epidemiological data suggest a correlation between the methyl content of diets and colorectal cancer. The current model is that an increased concentration of AdoMet stimulates DNA methyltransferase (DNMT) reactions, triggering hypermethylation and protecting the genome against global hypomethylation, a hallmark of cancer. Using an assay of active demethylation in HEK 293 cells, we show that AdoMet inhibits active demethylation and expression of an ectopically methylated CMV-GFP plasmid in a dose dependent manner. The inhibition of GFP expression is specific to methylated GFP; AdoMet does not inhibit an identical but unmethylated CMV-GFP plasmid. S-adenosylhomocysteine (AdoHcy), an analogue of AdoMet that differs by a single methyl residue, does not inhibit demethylation or expression of CMV-GFP. AdoMet but not AdoHcy inhibits methylated DNA binding protein 2/DNA demethylase (MBD2/dMTase) in vitro, suggesting that AdoMet directly inhibits MBD2/dMTase, and that the methyl residue on AdoMet is required for its interaction with MBD2/dMTase. We also demonstrate that AdoMet inhibits anchorage independent growth, an indicator of tumorigenesis, of HEK 293 and A549 cells. Taken together, our data support a new mechanism of action for AdoMet as an inhibitor of intracellular demethylase activity, which results in hypermethylation of DNA. The antitumorigenic effects of AdoMet are consistent with the hypothesis that demethylase activity is required for tumorigenesis.

### Materials and Methods

In vitro methylation of substrates—CMV-GFP (pEGFP-C1 from Clontech; Genbank accession #U55763) was methylated in vitro by incubating 10 μg of plasmid DNA with 12 units of SssI CpG methyltransferase (New England Biolabs) in the recommended buffer containing 800 μM AdoMet for 3 hours at 37° C. Twelve units of SssI and 0.16 μmol of AdoMet were then added and the reaction was further incubated for 3 additional hours. The methylated plasmid was recovered by phenol/chloroform extraction and ethanol precipitation, and complete methylation was confirmed by observing full protection from HpaII digestion.

Cell culture and transient transfections—Human embryonal kidney HEK 293 cells (American Type Culture Collection, under ATCC accession # CRL 1573) were plated at a density of 7.5×10⁴/well in a 6 well dish and transiently transfected with 80 ng of CMV-GFP (methylated or mock methylated) using the calcium phosphate precipitation method as described previously (17). 0.3 μM Trichostatin A (TSA) was added 24 hours post transfection. After an additional 24 h, cells were treated with or without various concentrations of AdoMet or AdoHcy (2-8 mM). Cells were harvested 72 hours post transfection. Each experiment was performed in triplicate, and experiments were performed several times using different cultures of HEK 293 cells.

Western blot analysis—Whole cell extracts were prepared using Radio Immuno Precipitation Assay buffer according to the Santa Cruz Biotechnology protocol, and protein concentrations were determined using the Bradford Reagent (BIO RAD). 2.5 μg of protein were resolved on a 12.5% SDS-polyacrylamide gel and then transferred to PVDF membrane (Amersham Pharmacia Biotech.). After blocking the nonspecific binding with 5% skim milk, GFP protein was detected using rabbit polyclonal IgG (Santa Cruz, sc-8334) at 1:500 dilution, followed by peroxidase-conjugated anti rabbit IgG (Sigma) at 1:5000, and enhanced chemiluminescence detection kit (Amersham Pharmacia Biotech). Blots were quantifed using NIH Image 1.62 software.

Southern Blot Analysis—DNA was extracted from HEK 293 cells using the DNeasy Tissue Kit (Quiagen). DNA was first digested with 50 units of EcoR1, followed by digestion with 20 units of either HpaII or MspI restriction enzymes. Samples were subjected to electrophoresis on a 1.5% agarose gel and then transferred to Hybond-N+ membrane (Amersham). Blots were probed with a ³²P-labeled CMV-GFP cDNA probe (AvaII-Cfr101 fragment) synthesized using a random priming labeling kit (Roche). Membranes were hybridized at 68° C. for 4-6 hours in a buffer containing 0.5 M sodium phosphate pH 6.8, 1 mM EDTA, 7% SDS, and 0.2 mg/ml herring sperm DNA. Following hybridization, the membranes were washed twice for 10 min in a 5% SDS, 0.04 M sodium phosphate pH 6.8, 1 mM EDTA solution, and then four times for 10 min in the same solution containing 1% SDS. The results of 3 independent experiments were quantified by densitometry (NIH Image 1.62). The demethylated, HpaII digested 529 bp fragment (U), and the methylated undigested DNA (M) were quantified for each sample, and the percent methylation was determined as [[M/(U+M)]*100.

AdoMet preparations for in vitro studies—AdoMet was prepared as a 50 mM solution in distilled water either by freeze drying a 32 mM AdoMet solution, which contained 10% ethanol and 5 mM sulfuric acid (New England Biolabs) immediately before use or by dissolving lyophilized powder (SIGMA) in distilled water. To control for possible nonspecific effects of remnants of the AdoMet storage buffer we freeze dried a solution of 5 mM sulfuric acid and 10% ethanol under the same conditions as done with the AdoMet solution and added distilled water after lyophilizing. AdoHcy was purchased from SIGMA and dissolved in distilled water at a 50 mM concentration.

Purification of recombinant MBD2/dMTase from SF9 cells—A fragment containing human MBD2/dMTase was excised from pCR2.1-dMTase (10) with BamHI and XhoI and transferred to the Baculovirus expression transfer vector pBlueBacHis2 C (Invitrogen). PBlueBacHis2 C-MBD2/dMTase and Bac-N-Blue viral DNA were co-transfected into the SF9 insect cell line, and recombinant viruses were isolated, identified and amplified according to the manufacturer's protocol (Invitrogen) with no modifications. High titer P3 viral stocks were used for infections. Insect SF9 cells were cultured in spinner flasks to a density of 2.5×10⁶ cells/ml in Grace's Insect Cell Culture Medium Supplemented (1×) from Life Technologies. For infection, 5×10⁶ cells were plated in 10 cm tissue culture plates (Sarstedt) and allowed to settle and attach for 30 minutes. The culture medium was removed and was replaced with 10 ml of the same medium containing MBD2/dMTase virus at a multiplicity of infection of 10. The cells were cultured with the virus for 5 days at 27° C. and were then harvested by scraping in cold phosphate buffered saline. Cell pellets from 10 plates were frozen and kept at −70° C. until they were used for enzyme purification. Frozen pellets were thawed in 5 ml of lysis buffer (10 mM Tris-HCl pH 8.0, 5 mM MgCl₂, 500 mM NaCl, 0.05% Tween 20, 10% glycerol and 10 mM imidazole) containing 1 μg/ml of the following protease inhibitors: aprotinin, leupeptin and pefablock. Protease inhibitors were added to all the solutions used in the purification. The homogenates were subjected to two cycles of freezing and thawing (5 minutes per step). DNA in the homogenate was sheered by passing through a 18.5 gauge needle 10 times. The extracts were then subjected to 15 cycles of sonication (10 seconds burst, 10 seconds gap per cycle at 20% of maximal output). The extracts were centrifuged at 10,000×g for 35 minutes. The supernatant was transferred into a fresh tube and was recentrifuged for additional 25 minutes at 15,000×g. The extract was filtered through a 5 micron filter to remove any particulate matter and the buffer was exchanged on a PD-10 buffer exchange column (Amersham Pharmacia) with buffer L (10 mM Tris-HCl pH 8.0, 10 mM MgCl₂) containing 50 mM NaCl. Recombinant MBD2/dMTase was partially purified by Q-sepharose (Pharmacia) ion exchange chromatography. Q-sepharose beads (1 ml of swollen beads) were washed extensively and preequilibrated with buffer L containing 50 mM NaCl and divided into 3 equal aliquots. The cell extracts were sequentially bound three times to the 3 aliquots of Q-sepharose beads in batch in 15 ml tubes by shaking gently on a Nutator for 45 minutes at 4° C. Following each binding step, the bound beads and unbound supernatant were separated by centrifugation for 2 minutes at 1000×g and the supernatant was transferred into a new tube and bound with new preequilibrated beads. The bound beads from the three binding steps were joined and resuspended in lysis buffer. The beads were washed in batch 4 times with 5 ml buffer L+50 mM NaCl. For each washing step, the beads were incubated with the wash solution for 15 minutes and were then separated from the wash supernatant by centrifugation for 2 minutes at 1000×g. Following washing, the proteins were eluted in batch (30 minutes per step) with a stepwise NaCl gradient in buffer L. Each elution step was analyzed for in vitro demethylase activity and for the presence of the recombinant His-tagged MBD2/dMTase by a Western blot analysis using the anti Xpress antibody from Invitrogen as previously described. MBD2/dMTase peak elution is at the 0.4 M NaCl step. No demethylase activity was observed in the same fractions prepared in a similar manner from uninfected SF9 cells.

Preparation of substrate DNA for in vitro demethylation assay—a. Methylation of substrate—Typically, 25 μg of DNA from micrococcus lysodeikticus (SIGMA, Type XI, highly polymerized) were methylated with M.SssI (60 u, New England Biolabs, NEB) and AdoMet (3.2 mM, NEB) in methylation buffer (NEbuffer2, NEB) in the presence of 50 mM EDTA for 3-4 hours at 37° C. Fresh AdoMet (3.2mM) and enzyme (40 u) were then added before incubating at 37° C. for additional 3-4 hours. To achieve complete methylation, methylation is repeated after AdoHcy, which is a product of the methylation reaction and inhibits DNA methylation, is removed using a Microcon 10 concentrator (Millipore). The degree of methylation was verified by methylation sensitive restriction enzyme analysis (Msp I-HpaII digestion) on aliquots of the reaction mixture. The DNA was purified by phenol-chloroform extraction (one part of either phenol or chloroform per three parts reaction mixture). Unincorporated nucleotides were removed by Nap5 gel filtration chromatography (Amersham Pharmacia) column. The Nap5 column was equilibrated with demethylation buffer (10 mM Tris-HCl, 5 mM MgCl₂, pH 7.0). DNA containing fractions were combined, concentrated on a Microcon 10, and subjected to a second Nap5 desalting column. DNA containing fractions were again concentrated as described above.

[α-³²P]dGTP labeling of DNA—We then prepared either methylated or unmethylated DNA that is ³²P labeled at G, the 3′ neighbor of the methylated C, as previously described (18) with the following modifications. 5 μg of either methylated or unmethylated DNA in 35 μl double distilled water were denatured and annealed to a hexanucleotide primer by boiling for 10 minutes in the presence of 3 μl random hexanucleotide mix (0.2 A₂₆₀) (Roche). The primed DNA was then subjected to template directed extension with the Klenow fragment of DNA polymerase I in the presence of labeled [α-³²P]-dGTP, either ^{methyl}dCTP (for methylated DNA) or dCTP for unmethylated DNA, dTTP and dATP. The 3′ phosphate of all the 5′ neighbors of G including either C or ^{methy}C is labeled by this procedure. The labeling was performed in polymerase buffer (50 mM NaCl, 6.6 mM Tris-HCl, 6.6 mM MgCl₂, 1 mM DTT, pH 7.4) with Klenow fragment I (10 u, Roche), ^{methyl}dCTP (1 mM) and dCTP (1 mM), respectively, dATP, dTTP (1 mM each) and [α-³²P]dGTP (50 μCi, Perkin Elmer Life Sciences) for 3 hours at 37° C. The reaction mixture was extracted with phenol and chloroform (one part of each per three parts reaction mixture). TCA precipitation (2 ml 10% trichloroacetic acid, 20 μg herring sperm DNA) of an aliquot showed typically 80-95% labeling efficiency (260,000-320,000 cpm per μl, total of 150 μl). The DNA was purified by eluting it twice from a Nap5 column and concentrating on a Microcon 10 as described above with the following variation: distilled water was used for prewashing and elution in the second column. The final concentration was 5 ng/μl and the specific activity was typically 8.0-8.8×10⁶ cpm per μg.

In vitro Demethylation Assay—A typical reaction mixture (50 μl) consisted of 25 ng of ³²P-prelabeled DNA (prepared as described above) incubated in demethylation buffer (10 mM Tris-HCl, 5 mM MgCl₂, pH 7.0) with the purified MBD2/dMTase (5 μl, ˜5 ng) for 24 hours at 37° C. in either the absence or presence of AdoMet and AdoHcy, respectively. The DNA was extracted from the enzyme by incubation in two volumes of DNA extraction buffer (10 mM Tris-HCl, 0.5 M NaCl, 1% SDS) containing 0.1 u proteinase K (Roche) at 50° C. for 2 hours. Subsequent phenol-chloroform extraction (one part of either phenol or chloroform per three parts of reaction volume) in the presence of tRNA (50 μg) as a carrier and ethanol precipitation with salt and 95% ethanol resulted in almost quantitative recovery of the input DNA. The DNA pellets were resuspended in distilled water (8 μl) and digested with microccocal nuclease to ³²P labeled 3′ mononucleotides as described elsewhere (27, 33). The labeled mononucleotides were separated by thin layer chromatography and visualized by autoradiography on a phosphorimager plate. The extent of demethylation was quantified from the image with the MCID-M4 software (Imaging Research Inc.).

Soft agar assay—HEK 293 and A549 cells were plated at a density of 7.5×10⁴ in a 6 well dish, and were treated the following day with either 4 mM AdoMet or AdoHcy for an additional 24 hours. For analysis of growth in soft agar, 3 ×10³ cells were seeded in triplicate onto a six well dish in 4 ml of complete medium containing 0.33% agar solution at 37° C. (34). Cells were fed with 2 ml of medium twice weekly. The assay was performed in triplicate, and colonies were counted 21 days after plating for A549 cells, and 7 days after plating for HEK 293 cells.

High throughput screening of demethylase inhibitors. The assay described here for demonstrating that AdoMet is a demethylase inhibitor in living cells is used for high-throughput screening of demethylase inhibitors. 5,000 HEK 293 cells are plated in a 96 well dish and are transfected with methylated CMV-GFP as described above. 24 hours following transfection all the wells are treated with 300 μM TSA. Increasing concentrations (10 nM-10 mM) of representative chemicals from libraries of chemical compounds are added to the wells. Following 96 hours the 96 wells plates are read in a fluorescent 96 well reader. Control wells and wells with inactive compounds show high fluorescence activity resulting from demethylation and expression of CMV-GFP. Wells that were treated with a putative inhibitor show dose dependent inhibition of fluorescence. The wells with hits are scanned for overt toxicity by live microscopy and trypan blue dye exclusion. DNA is extracted from the well as described here and the DNA is subjected to HpaII digestion as described above to validate that the putative compounds inhibited demethylation. Once this is validated the lead compounds is tested in in vitro demethylase assays as described above.

### Results

AdoMetinhibits TSA induced active demethylation of ectopically methylated and transiently transfected CMV-GFP in a dose dependent manner—There have been several reports demonstrating that exogenous administration of AdoMet leads to DNA hypermethylation (6, 8, 19). Similarly, other studies have shown that a decrease in dietary folate, or a depletion of intracellular AdoMet, results in DNA hypomethylation (2). However, it is not known whether AdoMet's effects on methylation are due to changes in DNMT or DNA demethylase activities.

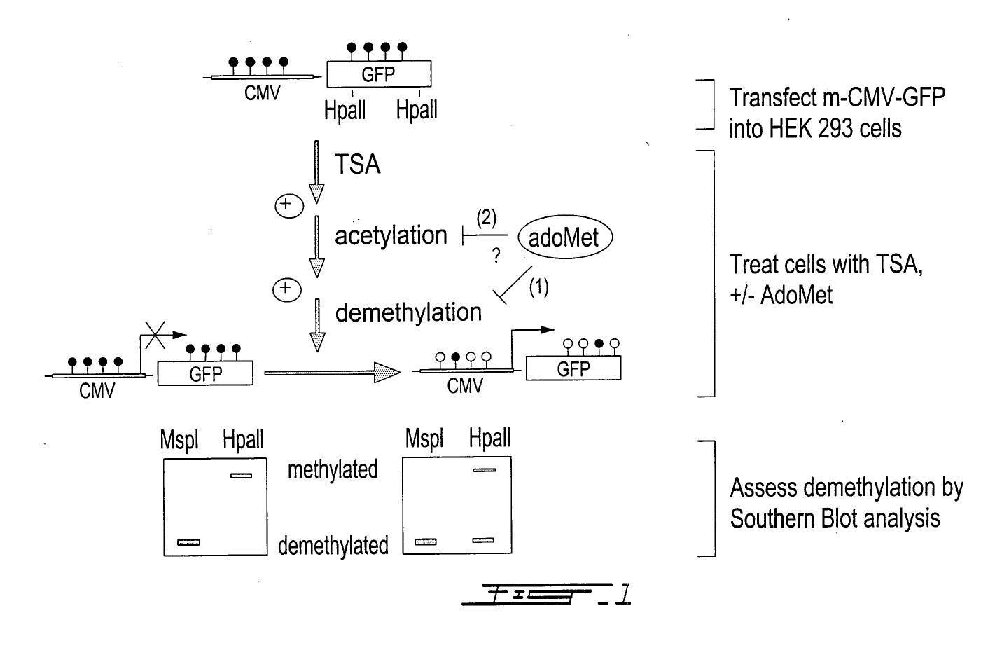

We utilized a previously described transient transfection based assay system (FIG. 1 and (15)) to study the effects of AdoMet on active demethylation of ectopically methylated DNA. In prior studies we have shown that in vitro methylated CMV-GFP reporter plasmid is actively demethylated 72 hours following transfection into HEK 293 cells when histone hyperacetylation is induced with TSA (15). Since CMV-GFP does not replicate nor is it de novo methylated in HEK 293 cells (15), this assay specifically measures active demethylation in a living cell.

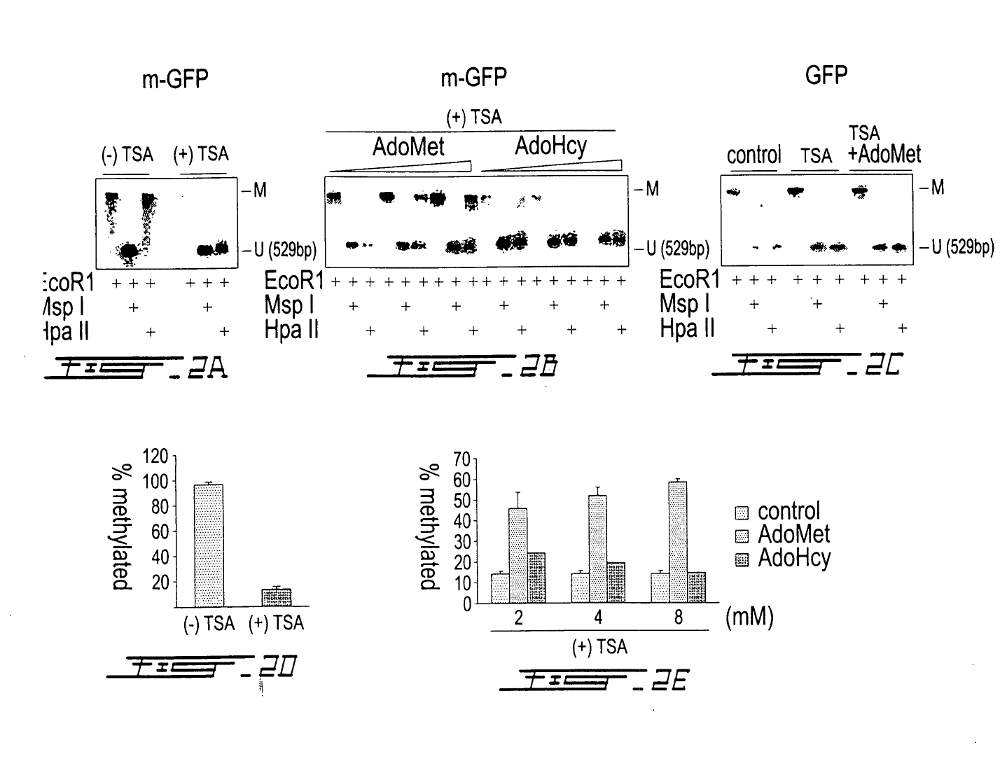

We first determined the effects of increasing doses of AdoMet, or its analogue AdoHcy, on the demethylation of methylated CMV-GFP (FIG. 2). DNA was isolated from HEK 293 cells transfected with methylated CMV-GFP DNA and treated with either TSA and AdoMet, or TSA and AdoHcy. DNA was first linearized with the Eco RI restriction enzyme, followed by digestion with MspI (which cleaves the sequence CCGG) or HpaII (which cleaves the sequence CCGG only when it is not methylated). As can be seen in FIG. 2A and D, the addition of TSA results in nearly complete demethylation of CMV-GFP by endogenous demethylase activity, as indicated by the complete HpaII digestion of CMV-GFP to the 529 bp fragment (U). Upon the addition of increasing concentrations of AdoMet (FIG. 2B and E), the percentage of methylated GFP remaining increases in a dose dependent manner, illustrated by the decrease in the ratio of the 529 bp HpaII fragment (U) to the undigested DNA (M). AdoHcy has an insignificant effect on the demethylation of CMV-GFP, indicating that the methyl moiety of AdoMet is required for inhibition of demethylation.

We then determined whether AdoMet stimulates de novo methylation of an identical unmethylated CMV-GFP substrate. FIG. 2C illustrates that unmethylated CMV-GFP, transfected under identical conditions, does not get de novo methylated, even in the presence of 8 mM AdoMet. This indicates that AdoMet does not cause an increase in DNMT activity on ectopic CMV-GFP. Thus, the likely mechanism by which AdoMet causes hypermethylation of CMV-GFP in comparison with the TSA treated control is by inhibiting its active demethylation by resident demethylases.

AdoMet reduces TSA induced expression of methylated CMV-GFP in a dose dependent manner, but has no effect on unmethylated CMV-GFP—A number of studies have shown that an increase in AdoMet inhibits gene expression (7, 8), however it is not clear whether AdoMet specifically affects genes whose methylation state it alters exclusively, or whether it has a non specific effect on gene expression. We took advantage of the CMV-GFP system described above to address this question. We determined whether AdoMet influences the expression of either methylated CMV-GFP, whose methylation state is affected by AdoMet, or unmethylated CMV-GFP, whose methylation state is not affected by AdoMet. HEK 293 cells were transiently transfected and treated with TSA and either AdoMet or AdoHcy, as described in the previous section. Extracts were then prepared and subjected to a Western blot analysis using an antibody directed against GFP protein.

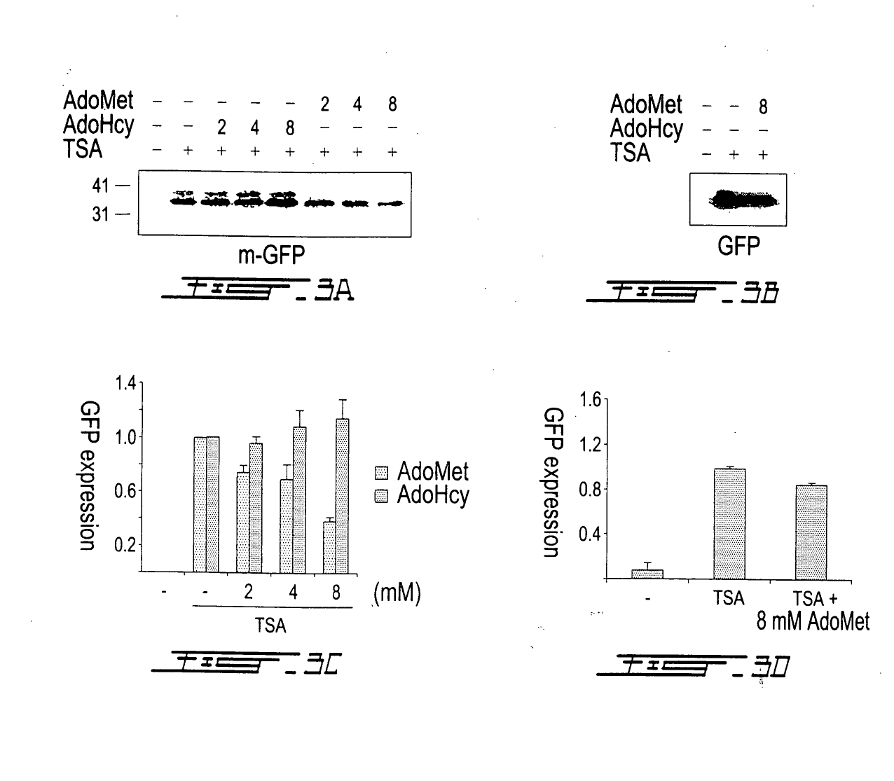

FIG. 3A and C illustrate that methylated CMV-GFP is completely repressed in untreated HEK 293 cells. This is as expected, since it is well documented that DNA methylation leads to gene silencing. The addition of TSA leads to a dramatic induction of GFP expression as expected from the complete demethylation following TSA treatment. Upon the addition of increasing amounts of AdoMet, GFP expression is decreased in a dose dependent fashion. AdoHcy has no significant effect on the expression of methylated GFP, consistent with its lack of effect of DNA demethylation.

Since our system measures expression and demethylation that is dependent on histone hyperacetylation, there are two possible mechanisms whereby AdoMet exerts its effects on demethylation (FIG. 1). AdoMet could directly inhibit a demethylase activity, or it could inhibit histone acetylation, which we have previously shown leads to an inhibition of demethylation (15). If the latter were true, then AdoMet should also inhibit the TSA induced expression of non methylated GFP, whose expression is induced by histone acetylation as well. FIG. 3B and D indicate that this is not the case, since AdoMet has no significant effect on the induction of non-methylated GFP by TSA. The fact that AdoMet specifically affects the expression of a methylated copy of CMV-GFP, and not an unmethylated copy, supports the model that AdoMet inhibits gene expression by directly inhibiting the active demethylation of methylated CMV-GFP.

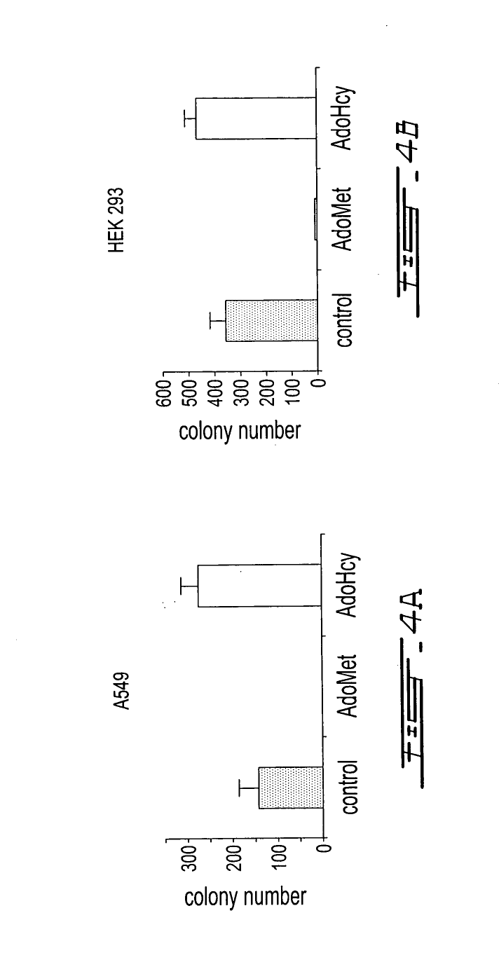

AdoMetinhibits the tumorigenic properties of two different transformed cells lines, A549 human lung carcinoma cells and HEK 293 human embryonal kidney cells—It is well documented that a correlation exists between reduced intracellular AdoMet (either as a consequence of decreased folate intake or pharmacological intervention) and an increase in cell proliferation and tumorigenesis (2, 19). However, there is no direct demonstration that AdoMet treatment of a cancer cell can revert its state of transformation. We therefore decided to test whether treatment with AdoMet could inhibit the transformation potential of HEK 293 cells, which were shown here to be responsive to AdoMet inhibition of demethylase. We also tested AdoMet effects on the transformed state of A549, a human lung non-small cell carcinoma cell line, to exclude the possibility that AdoMet effects are an idiosyncrasy of HEK 293 cells. Cells were treated for 24 hours with either AdoMet or AdoHcy at a concentration of 4 mM. An equal number of living cells were seeded onto soft agar to test their anchorage independent growth, an indicator of tumorigenesis in vitro. As shown in FIG. 4, both untreated HEK293 and A549 cells formed colonies in soft agar, which is characteristic of cancer cell lines. Remarkably, after only a single 24 hour treatment with AdoMet, no colonies are formed with A549 cells, and only a minimal number of colonies are observed with HEK 293 cells. In contrast, AdoHcy does not inhibit anchorage independent growth with either cell line, and even has a slight stimulatory effect. Since we have shown here that 4 mM AdoMet inhibits cellular demethylase activity in HEK 293 cells whereas AdoHcy has no effect at the same concentration, the inhibition of cell transformation is consistent with the hypothesis that hypomethylation is critical for tumorigenesis. AdoMet's chemoprotective effects against colorectal cancer might therefore be due to its inhibition of active demethylation.

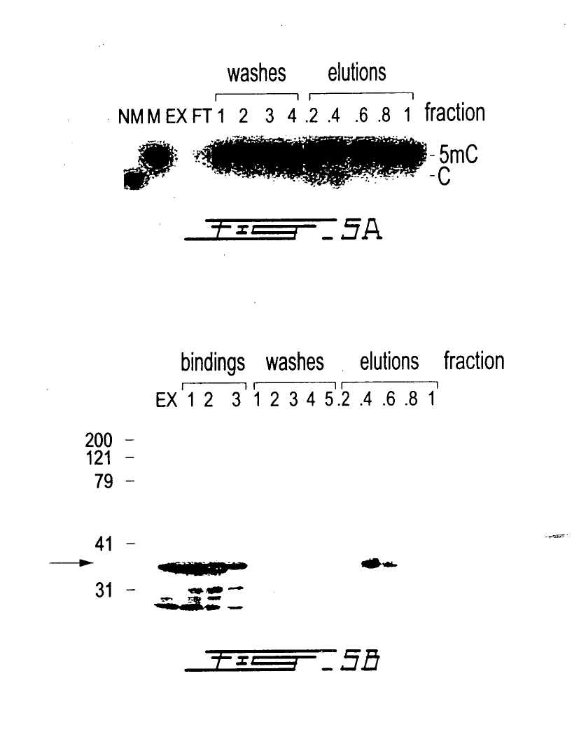

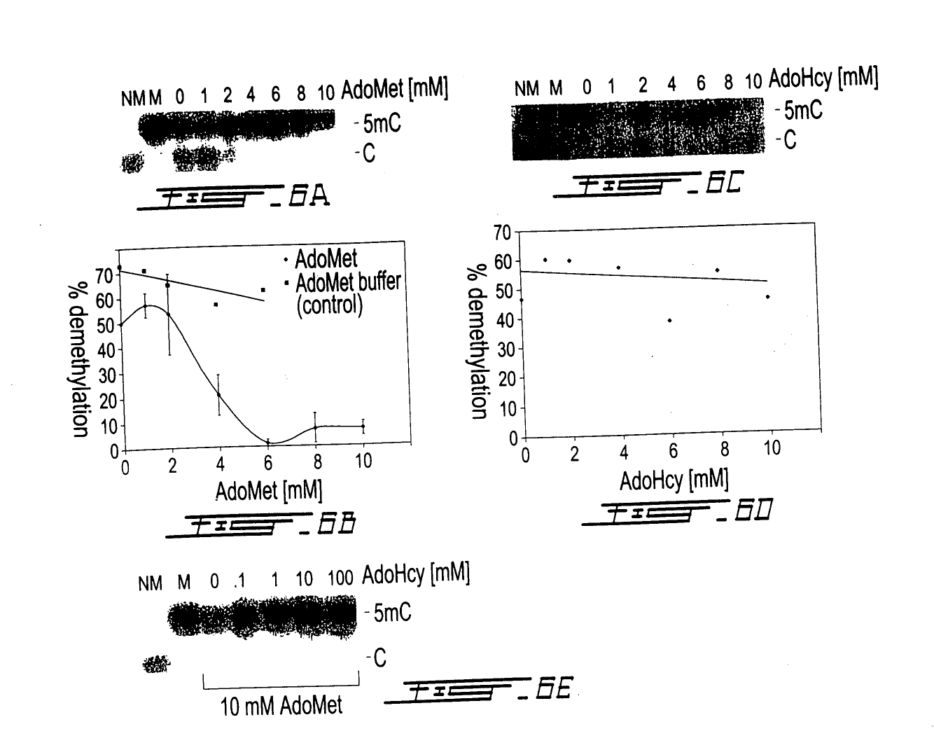

AdoMet but not AdoHcy inhibits Demethylation activity in vitro—In order to further confirm that the observed effect of AdoMet and AdoHcy is due to inhibition of active demethylation and not an indirect effect, in vitro studies with a recombinant MBD2/dMTase were performed (10). Although it is not certain whether MBD2/dMTase is responsible for the demethylation seen in HEK 293 cells, it is the only demethylase characterized thus far, and the in vitro experiment should test whether AdoMet can inhibit this demethylase activity. His tagged MBD2/dMTase was partially purified by chromatography on Q-Sepharose from nuclear extracts of SF9 cells infected with the recombinant MBD2/dMTase construct as described in materials and methods. Fractions were eluted with a stepwise gradient of NaCl and assayed for demethylation activity with a ³²P prelabeled methylated DNA from micrococcus lysodeikticus (FIG. 5A). Conversion of ^{methyl}dCMP to dCMP was almost exclusively detected with the 0.4 M NaCl fraction, although the 0.2 M fraction exhibited some low activity. This correlates with the peak presence of the His tagged recombinant MBD2/dMTase protein in this fraction as demonstrated by a Western blot analysis (FIG. 5B) using an anti-Xpress antibody. Next, we determined whether AdoMet inhibits the demethylation activity of MBD2/dMTase. The aforementioned DNA was incubated with MBD2/dMTase in the presence of increasing AdoMet concentrations. FIG. 6A presents the autoradiography of one representative experiment. Conversion of ^{methy}dCMP to CMP was greatly reduced at 2 mM AdoMet and abolished completely at concentrations higher than 4 mM. The dose curve in FIG. 6B is an average of quantitative autoradiography of 3 independent experiments. In order to exclude the possibility that traces of AdoMet storage buffer components (ethanol, sulfuric acid) inhibit demethylation, AdoMet storage buffer was freeze dried and added to control demethylation reactions in the same volumes as AdoMet. Inhibition of demethylation by this buffer is insignificant (less than 10%) as shown by the straight line in FIG. 6B in comparison to the 100% inhibition attained with AdoMet. Therefore, traces of buffer components do not significantly contribute to AdoMet's inhibition of MBD2/dMTase activity. Moreover, experiments with buffer free AdoMet showed an identical inhibitory effect (FIG. 6B, the curve is an average of experiments performed with both types of AdoMet preparations).

In contrast to AdoMet, no inhibition of demethylation occurred in the presence of increasing concentrations of AdoHcy (FIG. 6C). The curve in FIG. 6D is based on the quantitative autoradiography of the experiment in FIG. 6C. These results indicate that the small differences in the chemical structure (methyl group and positive charge on the sulfur) between AdoMet and AdoHcy are responsible for their different interaction with the MBD2/dMTase.

AdoMet and AdoHcy compete for binding to the catalytic site on DNMTs. It was therefore proposed that the ratio of AdoHcy to AdoMet determines DNMT activity as discussed in the introduction. AdoHcy inhibits DNMTs whereas increased AdoMet offsets this inhibition. We therefore determined whether a similar relationship applies to MBD2/dMTase. A competition experiment between AdoMet and AdoHcy is presented in FIG. 6E. Increasing concentrations of AdoHcy were added in the presence of an inhibitory concentration of AdoMet (10 mM), in a series of demethylation reactions. The results of this experiment illustrate that even a tenfold concentration excess of AdoHcy to AdoMet does not diminish inhibition of the demethylase reaction by AdoMet. This is consistent with the hypothesis that AdoMet has a higher affinity for MBD2/dMTase as compared with AdoHcy. Further studies are necessary to elucidate the mode of inhibition: whether AdoMet is a competitive inhibitor with the substrate DNA or an allosteric inhibitor as was demonstrated for Methylene Tetrahydrofolate Reductase. Furthermore, we do not know how MBD2/dMTase recognizes AdoMet on a structural basis.

### Discussion

The currently accepted mechanism for the effects of the methyl donor AdoMet on DNA methylation and tumorigenesis is founded on the assumption that the DNA methylation reaction is irreversible and defined exclusively by the DNMT. Taking advantage of our previously developed assay of demethylase activity in living cells (FIG. 1), we tested an alternative hypothesis: that AdoMet inhibits demethylase activity. If the steady state methylation status of DNA is maintained by an equilibrium of DNMT and demethylase activities (14), then inhibition of the demethylase side of the equilibrium should result in hypermethylation. Therefore, the reported DNA hypermethylation effects of exogenous AdoMet might be caused in part by inhibiting the level of demethylase activity in tumor cells. The main advantage of the system used in this paper is that it studies active demethylation exclusively, without interference from either replication dependent passive demethylation or de novo DNMT activities (15).

We show here that exogenous AdoMet inhibits TSA stimulated demethylation of ectopically methylated and transiently transfected CMV-GFP DNA (FIG. 2B, E). Since methylation inhibits the expression of CMV-GFP (15), inhibition of demethylation of CMV-GFP results in reduction of GFP protein expression (FIG. 3A, C), illustrating that AdoMet affects both demethylation of DNA and gene expression. This association of inhibition of demethylation and silencing of gene expression prompted us to rule out the possibility that AdoMet has a general, methylation-independent inhibitory effect on gene expression, or a general toxic effect, which also might result in inhibition of expression.

It is possible that AdoMet increases histone methyltransferase activity, resulting in hypermethylation of K9 on H3 histones, which has been shown to correlate with inhibition of acetylation. Inhibition of acetylation was shown to inhibit expression and demethylation of CMV-GFP (16). To address this alternative possibility, we measured in parallel the effects that AdoMet might have on methylated as well as unmethylated CMV-GFP plasmid, both transfected and treated with exogenous AdoMet under equivalent conditions. We first show that AdoMet treatment does not result in de novo methylation of unmethylated CMV-GFP (FIG. 2C). Thus, exogenous AdoMet does not stimulate DNA methylation as might be predicted by the current hypothesis of AdoMet's mechanism of action. Second, we show that exogenous AdoMet does not inhibit expression of unmethylated CMV-GFP under conditions where a clear inhibition of expression of methylated CMV-GFP is observed (FIG. 3B, D). Thus AdoMet specifically affects the expression of methylated genes. To our knowledge, this is the first demonstration that AdoMet specifically targets methylated DNA. This result also rules out the possibility that AdoMet exerts a general toxic effect on the cell. Our data therefore demonstrate that exogenous AdoMet specifically affects methylated DNA and prevents its expression. This most probably occurs by inhibiting an endogenous demethylase activity, resulting in hypermethylation of CMV-GFP and methylation-dependent repression.

We used the unmethylated analogue of AdoMet, AdoHcy, as a control. AdoHcy differs from AdoMet by a single methyl group. We show that AdoHcy has no effect on either gene expression (FIG. 3A, C) or demethylation (FIG. 2B, E). Taken together, these results indicate that both activities of AdoMet, inhibition of demethylation and inhibition of gene expression, are tightly associated and that they are both dependent on the methyl moiety in AdoMet.

We also demonstrate that AdoMet inhibits the tumorigenic potential of both HEK 293 and A549 cells at the same concentration that it inhibits demethylation and expression of methylated DNA (FIG. 4). This is consistent with epidemiological and clinical data, as well as with animal experiments using liver cancer models (2). Since we show that AdoMet specifically inhibits the expression of methylated genes, we suggest that part of the anti tumorigenic effects of AdoMet could be explained by the inhibition of demethylation and expression of genes that are required for anchorage independent growth. Further experiments will be required to identify the genes whose expression are inhibited by AdoMet and are required for anchorage independent growth and tumorigenesis.

In addition, we show that AdoMet directly inhibits recombinant MBD2/dMTase activity (FIG. 5) in a dose dependent manner using an in vitro assay (FIG. 6). This supports the hypothesis that AdoMet directly inhibits demethylase activity in living cells. AdoHcy does not inhibit MBD2/dMTase at the same concentrations (FIG. 6). Since an increase in intracellular AdoHcy was previously shown to be associated with hypomethylation, we tested the possibility that either AdoHcy stimulates MBD2/dMTase activity, or that it competes with AdoMet binding to MBD2/dMTase and relieves AdoMet inhibition. Our results suggest that AdoHcy does not interact with demethylase and that it has no effect on AdoMet inhibition of this enzyme in vitro. Our results support the conclusion that the methyl group in AdoMet is required for its interaction with MBD2/dMTase. Although our results demonstrate that exogenous AdoMet inhibits MBD2/dMTase in vitro and in living cells, there is no evidence that the intact AdoMet is the inhibitor. It is still possible that a metabolite of AdoMet is the active compound. Further experiments are required to test this possibility. Nevertheless, our experiments demonstrate that pharmacological administration of AdoMet inhibits active demethylation, alters gene expression, and inhibits tumorigenesis.

Further experiments are also required to demonstrate that the demethylase activity that is inhibited by AdoMet in HEK 293 cells is indeed MBD2/dMTase. It is possible that other demethylases are responsible for demethylating ectopically methylated DNA in HEK 293 cells. Nevertheless, our results provide a first proof of principle that AdoMet is an inhibitor of demethylase activity. Thus, in addition to its role as a cofactor of transmethylation reactions, AdoMet can also act as a regulator of DNA methylation metabolism by inhibiting demethylase activity.

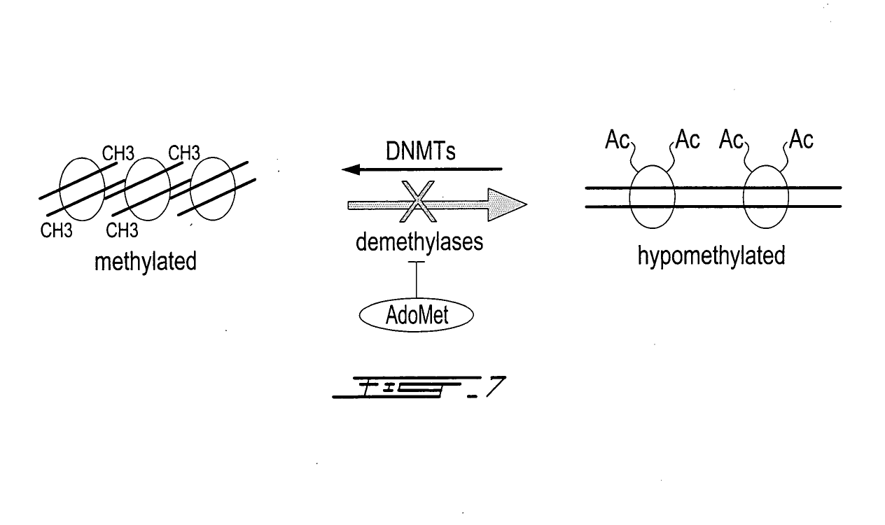

Our data further emphasize that the demethylase side of the methylation equilibrium has to be taken into account when dissecting the mechanism of action of drugs that modify the DNA methylation pattern. Based on our data, we suggest that AdoMet can alter DNA methylation patterns by inhibiting demethylase, which is expressed in some or most cells (FIG. 7). In this case, a reduction in the intracellular levels of AdoMet by methyl deficient diets removes this inhibition and increases the demethylase tone, resulting in active demethylation of DNA which could take place even in postmitotic tissue. Interestingly, AdoMet has recently been shown to inhibit the overall demethylation of a CG site in the 5′ of the myogenin gene during C2C12 differentiation (8). However, this report did not determine whether AdoMet stimulated DNMT or inhibited DNA demethylase.

The fact that AdoMet inhibits anchorage independent growth (FIG. 4) suggests that active demethylation might be especially important for tumorigenic or anchorage independent growth. This is consistent with the well-documented observations of global hypomethylation in cancer cells. There is evidence that AdoMet's tumor protective mechanism involves DNA methylation since this protection is removed when the animals are co treated with 5-azacytidine and AdoMet (6). In accordance with this hypothesis, we have recently shown that antisense inhibition of MBD2/demethylase inhibits tumorigenesis (20). It is tempting to speculate that certain genes that are required for anchorage independent growth might be inhibited by methylation and activated by a demethylase activity. Inhibition of the demethylase tone by AdoMet is proposed to result in silencing of these genes. Further experiments are obviously required to identify the genes that are suppressed by AdoMet and are required for anchorage independent growth. If AdoMet's mechanism of action in inhibiting tumorigenesis involves inhibition of demethylation, it would support the hypothesis that demethylation plays a causal role in tumorigenesis, and serve as a warning against using inhibitors of DNA methylation as anticancer agents.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### References

1. Poirier, L. A. (2002) J. Nutr. 132, 2336S-2339S
2. Pascale, R., Simile, M., De, M. M. and Feo, F. (2002) Alcohol 27, 193
3. Cheng, X. and Roberts, R. J. (2001) Nucleic Acids Res. 29, 3784-395.
4. Chiang, P. K., Gordon, R. K., Tal, J., Zeng, G. C., Doctor, B. P., Pardhasaradhi, K. and McCann, P. P. (1996) FASEB J. 10, 471-480
5. Ehrlich, M. (2002) J. Nutr. 132, 2424S-2429S
6. Pascale, R., Simile, M. M., Ruggiu, M. E., Seddaiu, M. A., Satta, G., Sequenza, M. J., Daino, L., Vannini, M. G., Lai, P. and Feo, F. (1991) Cancer Lett. 56, 259-265
7. Watson, W. H., Zhao, Y. and Chawla, R. K. (1999) Biochem. J. 342, 21-25
8. Fuso, A., Cavallaro, R. A., Orru, L., Buttarelli, F. R. and Scarpa, S. (2001) FEBS Lett. 508, 337-340
9. Ramchandani, S., Bhattacharya, S. K., Cervoni, N. and Szyf, M. (1999) Proc. Natl. Acad. Sci. USA 96, 6107-6112
10. Bhattacharya, S. K., Ramchandani, S., Cervoni, N. and Szyf, M. (1999) Nature 397, 579-583
11. Ng, H. H., Zhang, Y., Hendrich, B., Johnson, C. A., Turner, B. M., Erdjument-Bromage, H., Tempst, P., Reinberg, D. and Bird, A. (1999) Nat. Genet. 23, 58-61
12. Detich, N., Theberge, J. and Szyf, M. (2002) J. Biol. Chem. 277, 35791-35794
13. Szyf, M. (2001) Trends Pharmacol. Sci. 22, 350-354.
14. Szyf, M. (2001) Front. Biosci. 6, D599-609.
15. Cervoni, N. and Szyf, M. (2001) J. Biol. Chem. 276, 40778-4087.
16. Cervoni, N., Detich, N., Seo, S. B., Chakravarti, D. and Szyf, M. (2002) J. Biol. Chem.
17. Rouleau, J., Tanigawa, G. and Szyf, M. (1992) J. Biol. Chem. 267, 7368-7377
18. Szyf, M. and Bhattacharya, S. K. (2002) Methods Mol. Biol. 200, 155-161
19. Cai, J., Mao, Z., Hwang, J. J. and Lu, S. C. (1998) Cancer Res. 58, 1444-1450
20. Slack, A., Bovenzi, V., Bigey, P., Ivanov, M. A., Ramchandani, S., Bhattacharya, S., TenOever, B., Lamrihi, B., Scherman, D. and Szyf, M. (2002) J. Gene. Med. 4, 381-389

1. A DNA demethylase (dMTase) inhibitor which comprises s-adenosylmethionine, a metabolite of s-adenosylmethionine or a pharmaceutically acceptable salt thereof.

2. The DNA demethylase inhibitor of claim 1, wherein said DNA demethylase is methylated DNA binding protein 2/DNA demethylase (MBD2/dMTase).

3. The DNA demethylase inhibitor of claim 2, wherein said DNA demethylase comprises an amino acid sequence set forth in SEQ ID NOS: 1-4.

4. An antitumorigenic agent which comprises s-adenosylmethionine, a metabolite of s-adenosylmethionine or a pharmaceutically acceptable salt thereof.

5. A DNA hypermethylating agent which comprises s-adenosylmethionine, a metabolite of s-adenosylmethionine or a pharmaceutically acceptable salt thereof.

6. A method for inhibiting a DNA demethylase (dMTase) comprising the steps of:
a) incubating a mixture comprising ³²P-prelabeled DNA, a demethylation buffer, purified MBD2/dMTase and s-adenosylmethionine and/or s-adenosylhomocysteine;
b) extracting said DNA from said MBD2/dMTase;
c) digesting said DNA with a microccocal nuclease, whereby obtaining ³²P-labeled 3′ mononucleotides;
d) purifying said labeled mononucleotides obtained in step (c); and
e) quantifying demethylation of said DNA.

7. The method of claim 6, wherein said DNA demethylase is methylated DNA binding protein 2/DNA demethylase (MBD2/dMTase).

8. The method of claim 7, wherein said DNA demethylase comprises an amino acid sequence set forth in SEQ ID NOS: 1-4.

9. A method for inhibiting a tumorigenesis or a tumorigenic potential of cells of a patient which comprises the step of administering to said patient a medicament comprising s-adenosylmethionine, a metabolite of s-adenosylmethionine or a pharmaceutically acceptable salt thereof.

10. The method of claim 9, wherein said medicament is administered intravenously, intramuscularly, sub-cutaneously, transdermally, or per os.

11. The method of claim 9, wherein said cells are selected from the group consisting of A549 human lung carcinoma cells, and HEK 293 human embryonal kidney cells.

12. The method of claim 11, wherein said patient is a mammalian.

13. The method of claim 12, wherein said mammalian is a human.

14. A method for inhibiting tumor cell growth of a patient which comprises the step of administering to said patient a medicament comprising s-adenosylmethionine, a metabolite of s-adenosylmethionine or a pharmaceutically acceptable salt thereof.

15. The method of claim 14, wherein said tumor is a liver tumor.

16. The method of claim 15, wherein said patient is a mammalian.

17. The method of claim 16, wherein said mammalian is a human.

18. An assay for identifying a DNA demethylase inhibitor (dMTase) which comprises the steps of:
a) incubating a mixture comprising 32P-prelabeled DNA, a demethylation buffer, purified MBD2/dMTase and s-adenosylmethionine and/or s-adenosylhomocysteine;
b) extracting said DNA from said MBD2/dMTase;
c) digesting said DNA with a microccocal nuclease, whereby obtaining ³²P-labeled 3′ mononucleotides;
d) purifying said labeled mononucleotides obtained in step (c); and
e) quantifying demethylation of said DNA.

19. The assay of claim 18, wherein said DNA demethylase is methylated DNA binding protein 2/DNA demethylase (MBD2/dMTase).

20. The assay of claim 19, wherein said DNA demethylase comprises an amino acid sequence set forth in SEQ ID NOS: 1-4.

21. The assay of claim 18, wherein said buffer is 10 mM Tris-HCl and 5 mM MgCl₂.

22. The assay claim 21, wherein said buffer has a pH of 7.0.

23. The assay of claim 18, wherein in step (b), DNA is extracted from MBD2/dMTase by incubating a composition comprising said DNA, an extraction buffer and a proteinase K.

24. The assay of claim 23, wherein in step (b), after said incubation, DNA is further extracted using a first organic solvent and tRNA, and said DNA is then precipitated using a salt and a second organic solvent.

25. The assay of claim 24, wherein said first organic solvent is phenol or chloroform.

26. The assay of claim 24, wherein said second organic solvent is ethanol.

27. The assay of claim 26, wherein in step (d), said mononucleotides are purified by thin layer chromatography or column chromatography.

28. The assay of claim 26, wherein in step (e), said labeled mononucleotides are visualized by autoradiography on a phosphorimager plate.

29. A method of high throughput screening of DNA demethylase (dMTase) inhibitors in living cells comprising the steps of:
a) transfecting HEK 293 cells in a 96 wells dish with methylated CMV-GFP;
b) treating wells with 300 μM TSA and subsequently adding inhibitors for screeing;
c) reading fluorescence to determine inhibition of. DNA demethylase, wherein a putative inhibitor shows dose dependent inhibition of fluorescence.

30. The assay of claim 29, which further comprises a step, conducted after step (c), extracting DNA from wells with low fluorescence to validate putative inhibitor activity.


SEQUENCE LISTING (4 sequences)

SEQ ID NO: 1 - protein, 411 aa, Unknown
  DEMETHYLASE OR FRAGMENT THEREOF
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 2 - protein, 291 aa, Unknown
  DEMETHYLASE OR FRAGMENT THEREOF
MERKRWECPALPQGWEREEVPRRSGLSAGHRDVFYYSPSGKKFRSKPQLARYLGGSMDLSTFDFRTGKMLMSKMNKSRQRVRYDSSNQVKGKPDLNTALPVRQTASIFKQPVTKITNHPSNKVKSDPQKAVDQPRQLFWEKKLSGLNAFDIAEELVKTMDLPKGLQGVGPGCTDETLLSAIASALHTSTMPITGQLSAAVEKNPGVWLNTTQPLCKAFMVTDEDIRKQEELVQQVRKRLEEALMADMLAHVEELARDGEAPLDKACAEDDDEEDEEEEEEEPDPDPEMEHV

SEQ ID NO: 3 - protein, 414 aa, Unknown
  DEMETHYLASE OR FRAGMENT THEREOF
MRAHPGGGRCCPEQEEGESAAGGSGAGGDSAIEQGGQGSALAPSPVSGVRREGARGGGRGRGRWKQAARGGGVCGRGRGRGRGRGRGRGRGRGRGRPQSGGSGLGGDGGGGAGGCGVGSGGGVAPRRDPVPFPSGSSGPGPRGPRATESGKRMDCPALPPGWKKEEVIRKSGLSAGKSDVYYFSPSGKKFRSKPQLARYLGNAVDLSSFDFRTGKMMPSKLQKNKQRLRNDPLNQNKGKPDLNTTLPIRQTASIFKQPVTKFTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEVDIDMDSGDEA

SEQ ID NO: 4 - protein, 285 aa, Unknown
  DEMETHYLASE OR FRAGMENT THEREOF
MERKRWECPALPQGWEREEVPRRSGLSAGHRDVFYYSPSGKKFRSKPQLARYLGGSMDLSTFDFRTGKMLMNKMNKSRQRVRYDSSNQVKGKPDLNTALPVRQTASIFKQPVTKITNHPSNKVKSDPQKAVDQPRQLFWEKKLSGLSAFDIAEELVRTMDLPKGLQGVGPGCTDETLLSAIASALHTSTLPITGQLSAAVEKNPGVWLNTAQPLCKAFMVTDDDIRKQEELVQQVRKRLEEALMADMLAHVEELARDGEAPLDKACAEEEEEEEEEEEEPEPERV